(19) United States
(12) Patent Application Publication
(10) Pub. No.: US 2025/0195704 A1
(43) Pub. Date: Jun. 19, 2025

(54) DISINFECTION DEVICE AND METHOD FOR THE DISINFECTION OF LIQUIDS AND GASES

(71) Applicant: COMPACT LASER SOLUTIONS GMBH, Berlin (DE)

(72) Inventor: Andreas KUNTZE, Berlin (DE)

(73) Assignee: COMPACT LASER SOLUTIONS GMBH, Berlin (DE)

(21) Appl. No.: 18/554,806

(22) PCT Filed: Apr. 11, 2022
(86) PCT No.: PCT/EP2022/059567

§ 371 Date: May 13, 2024

(30) Foreign Application Priority Data
Apr. 12, 2021 (DE) 10 2021 109 004.0

(51) Int. Cl. A61L 2/10; A61L 9/20; C02F 1/32

(52) U.S. Cl. CPC A61L 2/10; A61L 9/20; C02F 1/325; A61L 2202/11; A61L 2202/122; A61L 2209/12; C02F 2201/3226; C02F 2201/328; C02F 2301/028; C02F 2303/04

(57) ABSTRACT

The invention relates to a process for the disinfection of liquids or gases with the process steps: Creating a radiation barrier in a disinfection chamber, introducing a gas and/or a liquid flow into the disinfection chamber, guiding the gas and/or liquid flow through a plurality of volume ranges of the disinfection chamber and discharging the gas and/or liquid flow from the disinfection chamber, wherein the volume ranges in the disinfection chamber are formed by the radiation barrier, a chamber wall and a flow guiding surface of a flow guiding element and the volume ranges are completely separated from each other by the chamber wall, the radiation barrier and the flow guiding surface, as well as a device for carrying out the method.

The invention relates to a process for the disinfection of liquids or gases, comprising the steps of: creating a radiation barrier in a disinfection chamber, introducing a gas and/or liquid flow into the disinfection chamber, passing the gas and/or liquid flow through a plurality of volume ranges of the disinfection chamber and discharging the gas and/or liquid flow from the disinfection chamber, and a device for carrying out the process.

### STATE OF THE ART

Ultraviolet light is a well-known disinfectant for surfaces, liquids and gases. Gas discharge lamps such as low-pressure mercury vapour lamps, xenon lamps, eximer lamps, UV LEDs and, to a very small extent, UV lasers are used as light sources. The disinfection of the liquid or gas itself can take place in special containers through which the liquid or gas flows.

Such a process for the disinfection in particular of liquids (water), but also of gases, as well as a corresponding device is shown in U.S. Pat. No. 4,661,264. A pulsed laser beam is irradiated into the container through which the liquid flows. The liquid flow intersects the laser beam several times during the process. The laser beam is basically arranged in a fixed position on the container.

One disadvantage of the container presented here is the presence of areas where the liquid does not intersect the laser beam, i.e. is not impacted by the disinfecting laser beam thus is not disinfected. Another disadvantage of another configuration of the presented container is the low energy input of the laser beam acting on the liquid. Thus, for thorough disinfection, the laser beam must have a high power density with simultaneously high average power in pulsed operation. To generate high power densities, it is necessary to operate the laser in the transverse fundamental mode with a correspondingly low divergence angle. In addition, the provision of the fundamental mode operation is of decisive importance for the generation of a wide variety of beam shapes. Optimisation of the disinfection process can be ensured through targeted changes to the transverse mode.

The following possibilities can be used as an example:
Top Hat Beam Shaper
Homogenisers/diffusers
Elliptical diffusers
Vortex lenses.
Diffractive axicon elements
DOEs for structured light

U.S. Pat. No. 10,370,267 B2 discloses a container for disinfecting liquids, in which the container has one or more radiation sources that emit radiation in the UV range. The liquid flows through the container, which has diffusely reflecting surfaces that guide the UV radiation through the liquid.

The use of PTFE, as described in the patent, enables a reflectance of approximately 97%. However, the decontamination efficiency is reduced by the diffuse multiple reflection of the diverging UV LEDs in the reaction chamber. The reflection efficiency decreases very strongly cumulatively. In addition, the reflector of the container is also subject to a very rapid, serious impairment of the reflectivity, which can occur after only a few hours, depending on the water quality, due to deposits of dissolved ions such as calcium, sodium and iron etc. and general particles of dirt. Efficient and safe decontamination of pathogens is thus rapidly limited. Regular replacement of components results in corresponding maintenance cycles and maintenance costs.

It is therefore an object of the present invention to provide a method for disinfecting liquids or gases with which disinfection can be carried out reliably, cost-effectively and energy-efficiently, and which is robust with respect to deposits on the surfaces of the reaction chamber.

It is also an object of the invention to provide a diffusion reactor for the disinfection of liquids or gases, with which disinfection can be carried out reliably, cost-effectively and energy-efficiently.

The task is solved by means of the process for disinfecting liquids or gases according to claim 1. Advantageous embodiments of the invention are set out in the dependent claims.

The process for disinfecting liquids or gases according to the invention has four process steps: In the first process step, a radiation barrier is created in a disinfection chamber. The UV radiation from the radiation source is shaped by optical components in such a way that UV radiation impinges on a surface and/or a volume within the disinfection chamber. In the second process step, a liquid and/or a gas flow is introduced into the disinfection chamber. For this purpose, the disinfection chamber has an inlet on one side. In the third process step, the liquid and/or gas flow is passed through several volume ranges of the disinfection chamber. In the fourth process step, the liquid and/or gas flow is channelled out of the disinfection chamber. For this purpose, the disinfection chamber has an outlet on the side opposite to the inlet.

According to the invention, the volume ranges of the disinfection chamber are formed by the radiation barrier, a wall of the disinfection chamber, and a flow guiding surface of a flow guiding element. The individual volume ranges of the disinfection chamber are completely separated from each other by the radiation barrier, by a wall of the disinfection chamber, and by a flow guiding surface of a flow guiding element. This advantageous arrangement allows the substance to be disinfected to pass through the Radiation barrier several times. This increases the exposure time of the UV radiation to the substance to be disinfected.

In a further embodiment of the invention, the flow guiding element is arranged as a separate component in the disinfection chamber. Advantageously, the disinfection chamber has several flow guiding elements of the same type. The disinfection chamber can be enlarged by installing further flow guiding elements of the same type. In addition, the manufacturing costs of a flow guiding element are reduced due to possible series production.

In another embodiment of the invention, the flow guiding element is attached to a wall of the disinfection chamber. A flow guiding element is therefore advantageously arranged on one side of the disinfection chamber.

In an advantageous embodiment of the invention, the gas and/or liquid flow is guided through a f low guiding element with several flow guiding surfaces and/or through the flow guiding surfaces of several flow guiding elements. The gas or liquid flow is guided through the flow guiding surfaces within the disinfection chamber. This increases the exposure time of the UV radiation to the substance to be disinfected.

In a further embodiment of the invention, the flow guiding surface has a curved surface along which the gas and/or liquid flow is guided. This increases the exposure time of the UV radiation to the substance to be disinfected.

In another embodiment of the invention, the radiation of the radiation barrier is irradiated into the disinfection chamber through a window that is transparent to the radiation. The transparent window may be arranged at a Brewster angle to allow polarised radiation to enter the Disinfection chamber. The window may also be designed to be movable to control the entry of the radiation barrier. Furthermore, the exit window can also be arranged at a brewster angle to the beam direction.

The use of a brewster window made of quartz glass is advantageous because a complex, cost-intensive anti-reflective coating can be dispensed with, which is also subject to rapid degradation due to UV radiation. The transmission of polarised light through a window at the brewster angle is almost loss-free. In the event of any degradation caused by UV light or general soiling of the brewster window, it can be advanced by an appropriate amount to ensure the undamaged passage of light. A rotating circularly symmetrical window arranged at a brewster angle would be advantageously applicable as another embodiment.

In another embodiment of the invention, the radiation barrier is created in a width B that is greater than or equal to an inner diameter D of the disinfection chamber. This ensures that the gas or liquid flow deflected by the flow guiding elements must flow through the radiation barrier, advantageously several times. This increases the exposure time of the UV radiation to the substance to be disinfected.

In a further embodiment of the invention, the gas and/or liquid flow is guided several times through the radiation barrier. This ensures that the gas or liquid flow deflected by the Flow guiding elements must flow through the radiation barrier, advantageously several times. This increases the exposure time of the UV radiation to the substance to be disinfected.

In a further embodiment of the invention, the radiation barrier is redirected. Using suitable optical components, the radiation barrier is redirected in such a way that it is re-emitted into the disinfection chamber at an angle to the primary radiation barrier (secondary radiation barrier). This also increases the exposure time of the UV radiation to the substance to be disinfected. It is also possible to redirect the Radiation barrier to introduce it into further disinfection chambers. Thus, a number of disinfection chambers are operated by means of one radiation source.

In a further development of the invention, several radiation barrier sections are created in the disinfection chamber as a result of the deflection of the radiation barrier. The gas or liquid flow is guided several times through the radiation barrier sections due to the flow guiding elements. This also increases the exposure time of the UV radiation to the substance to be disinfected.

In another embodiment of the invention, the radiation of the radiation barrier is laser radiation. UV light, particularly the UV-C spectrum, is suitable for effectively deactivating bacteria and viruses. UV-C in this context is the spectrum between 280 and 100 nanometres (nm) wavelength. In contrast to the UV-A (380 nm-315 nm) and UV-B range (315 nm-280 nm), this range of UV light is almost completely absorbed by oxygen in the atmosphere and does not reach the earth's surface under natural conditions. Within the UV-C spectrum, a further distinction must be made according to the biological effect. At 280 nm, proteins in particular-especially the amino acid tryptophan-absorb light, which leads to denaturation. At 265 nm, nucleic acids are damaged, especially by dimerisation of the base thymine. In the range of 245 nm, mainly nucleic acids absorb, while there is an absorption minimum for proteins in the range, which allows the technical application of a narrow frequency spectrum, e.g. for the disinfection of protein solutions.

In a further embodiment of the invention, the entire gas and/or liquid flow is guided through several volume ranges of the Disinfection chamber. This has the advantage that the entire gas and/or liquid flow is guided through at least one radiation barrier, thus ensuring that the entire gas and/or liquid flow has been disinfected when it leaves the disinfection reactor.

The task is also solved by means of a disinfection reactor for the disinfection of liquids and/or gases.

The disinfection reactor for disinfecting liquids and/or gases according to the invention has a disinfection chamber which has an inlet and an outlet for the substance to be disinfected. Furthermore, the disinfection chamber has a chamber wall. The disinfection chamber according to the invention further comprises a flow guiding element. The disinfection chamber additionally comprises a radiation element which is suitable for creating a radiation barrier in the disinfection chamber.

According to the invention, the disinfection chamber has several volume ranges which are completely separated from each other by the radiation barrier, the flow guiding element and the chamber wall. The gas or liquid flow is guided several times through the radiation barrier due to the flow guiding element. This increases the exposure time of the UV radiation to the substance to be disinfected.

In another embodiment of the invention, the flow guiding element has a flow guiding surface. The gas or liquid flow is guided through the flow guiding surfaces inside the disinfection chamber. This increases the exposure time of the UV radiation to the substance to be disinfected.

In a further development of the invention, the flow guiding surface has a curved surface. The gas or liquid flow is deflected at the flow guiding surface in such a way that it crosses/intersects the radiation barrier. The gas or liquid flow is guided through the flow guiding surfaces inside the disinfection chamber. This increases the exposure time of the UV radiation to the substance to be disinfected.

In a further embodiment of the invention, the disinfection chamber has several flow guiding elements and/or flow guiding surfaces. The disinfection chamber can be enlarged by installing further flow guiding elements of the same type. In addition, the manufacturing costs of a flow guiding element are reduced due to a possible series production.

In another embodiment of the invention, each volume range is separated from an adjacent volume range by at least one radiation barrier. The gas or liquid flow is guided several times through the radiation barrier sections due to the flow guiding elements. This also increases the exposure time of the UV radiation to the substance to be disinfected.

In a further embodiment of the invention, the disinfection chamber has several radiation barriers. Using suitable optical components, the radiation barrier is deflected in such a way that it is re-emitted into the disinfection chamber at an angle to the primary radiation barrier (secondary radiation barrier). This also increases the exposure time of the UV radiation to the substance to be disinfected.

In another embodiment of the invention, the disinfection chamber has a transparent window for radiation generated by the radiation element. The transparent window may be arranged at a brewster angle to allow polarised radiation to enter and/or exit the disinfection chamber. The window may also be designed to be slidable to prevent the radiation barrier from entering.

In a further development of the invention, the disinfection chamber has elements for deflecting the radiation generated by the radiation element. The radiation barrier can, for example, be introduced into further disinfection chambers. Thus, a plurality of disinfection chambers are operated by means of one radiation source.

In an advantageous design of the invention, the inlet is separated from the outlet by several volume ranges. The gas or liquid flow is guided several times through the radiation barrier sections due to the flow guiding elements. This also increases the exposure time of the UV radiation to the substance to be disinfected.

Examples of embodiments of the disinfection reactor according to the invention and of the process according to the invention for disinfecting liquids or gases are shown schematically in simplified form in the drawings and are explained in more detail in the following description.

### Showing:

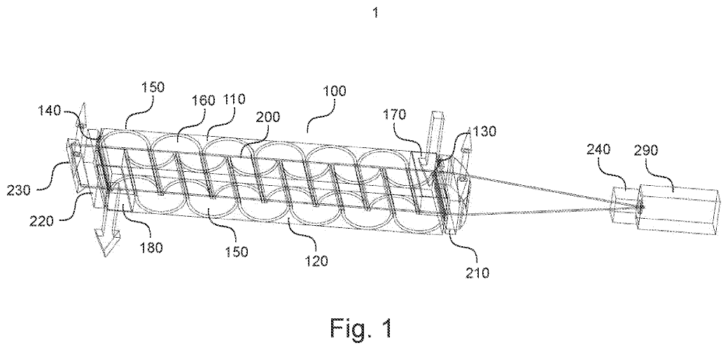

FIG. 1: Disinfection reactor with radiation source

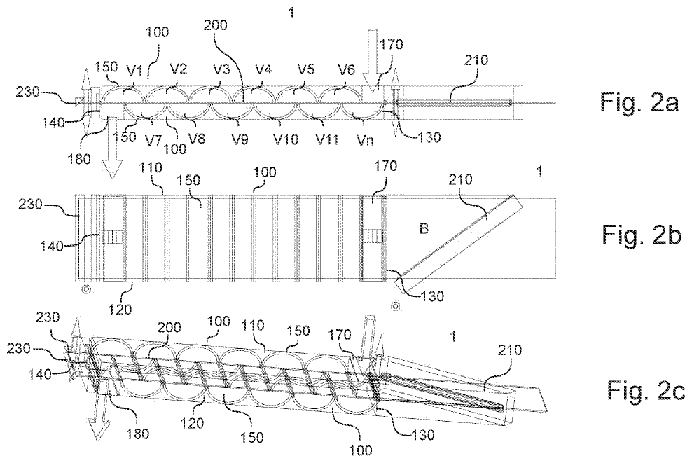

FIG. 2a: Disinfection reactor with concave flow guiding elements-side view

FIG. 2b: Disinfection reactor with concave flow guiding elements-top view

FIG. 2c: Disinfection reactor with concave flow guiding elements-perspective view

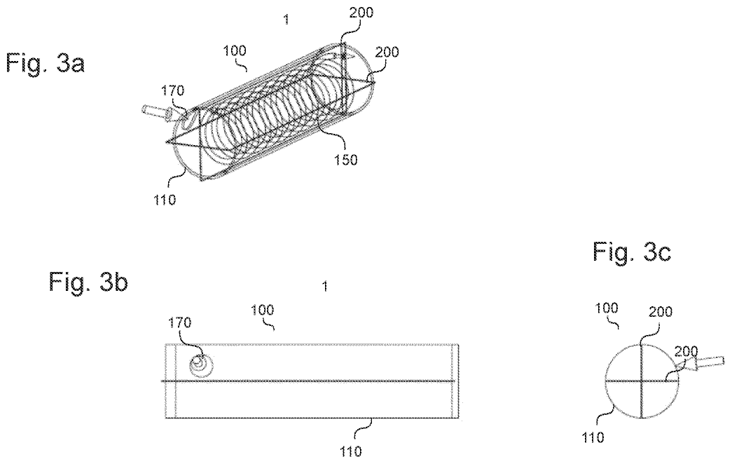

FIG. 3a)-c): Disinfection reactor as vortex chamber with two radiation barriers

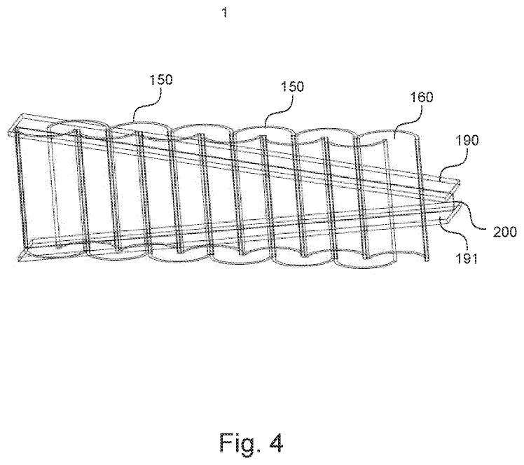

FIG. 4: Disinfection reactor for diverging radiation barrier

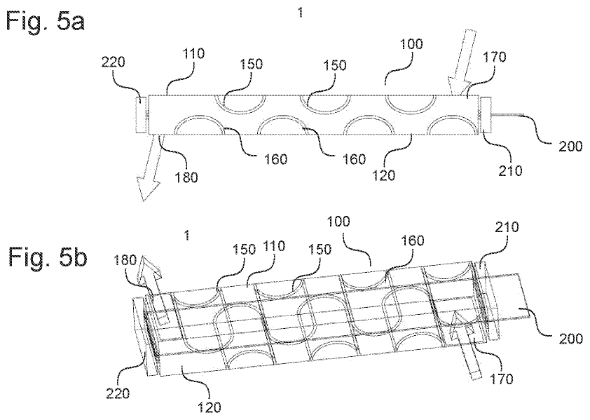

FIG. 5a: Disinfection reactor with convex flow guiding elements-side view

FIG. 5b: Disinfection reactor with convex flow guiding elements-perspective view

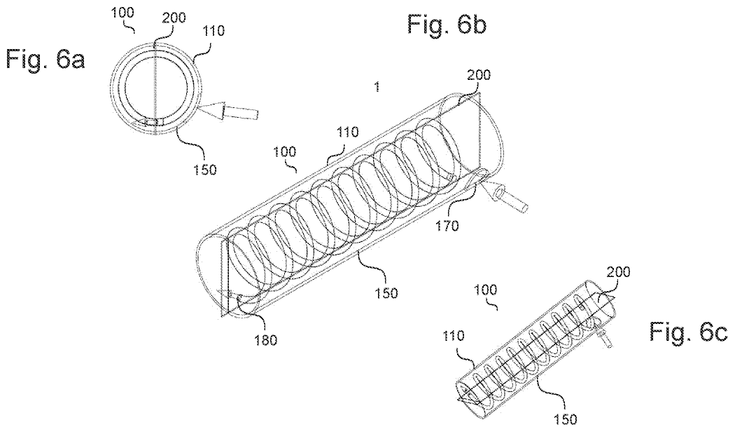

FIG. 6a)-c): Disinfection reactor as vortex chamber with a radiation barrier

FIG. 1 shows an embodiment of a disinfection reactor 1 for disinfecting gases and/or liquids. The disinfection reactor 1 has the disinfection chamber 100, which is bounded by side walls 120, 130 at the sides, by walls 140, 150 at the ends and by walls on top and bottom sides (not shown). The disinfection chamber 100 has openings 170, 180 suitable for the entry and exit of the substance (gas or fluid) to be disinfected. The disinfection chamber 100 has two rows of flow guiding elements 150 arranged parallel to each other, which are arranged opposite each other in such a way that the two rows are displaced relative to each other by half the width of a flow guiding element 150. The flow guiding elements 150 are arranged as separate components on the walls 110, 120 in the disinfection chamber 100.

The flow guiding elements 150 each have a flow guiding surface 160 on the concavely curved side, along which the gas or liquid flow is guided. A flow guiding element 150 essentially has the shape of a halved cylinder. Other shapes of flow guiding elements 150 are also possible, e.g. a sawtooth shape. All that is required is a suitable shape so that the gas or liquid flow is deflected around the central axis of the disinfection chamber 100.

The beam source 290 is a laser that generates radiation with a wavelength in the UV range, preferably in the UV-C range (280 nm to 100 nm). The radiation from the laser 290 is irradiated into the disinfection chamber 100 through a window 210 that is transparent to the radiation. The laser 290 has a beam shaping element 240 which is used to expand the laser beam into a plane. The laser beam enters the disinfection chamber 100 through a window 210. The laser beam forms a radiation barrier 200 in the disinfection chamber 100, which runs along the longitudinal axis in the central axis of the disinfection chamber 100.

On the side of the disinfection chamber 100 opposite the entry window 210, the disinfection chamber 100 has an exit window 220. The laser barrier 200 can therefore be guided into further disinfection chambers 100, e.g. by means of a deflector optics 230. The entry window 210, like the exit window 220, can be moved perpendicular to the plane of the laser barrier 200.

Radiation barrier 200 is constructed in disinfection chamber 100 to disinfect a gas or liquid. In this embodiment, the radiation barrier 200 runs along the longitudinal axis in the central axis of the disinfection chamber 100. The substance to be disinfected is directed into the disinfection chamber 100 via the inlet 170 in such a way that a continuous gas or liquid flow is formed in the disinfection chamber 100. Optimally, the gas or liquid flow has a constant flow rate (volume/time unit) during the disinfection process. The substance to be disinfected leaves the disinfection chamber 100 via outlet 180. Between inlet 170 and outlet 180, the gas or liquid flow is guided through the laser barrier 200 by the flow guiding surfaces 160 of the flow guiding elements 150, advantageously in several volume ranges formed by the flow guiding elements 150, which increases the time that the laser barrier 200 can act on the substance to be disinfected.

A variant of the disinfection reactor 1 according to the invention for the disinfection of gases and/or liquids is shown in FIG. 2. The disinfection chamber 100 corresponds to the disinfection chamber 100 described in FIG. 1 (FIG. 2a, 2b). The radiation of the laser 290 is irradiated into the disinfection chamber 100 through a window 210 which is transparent to the radiation. The collimator optics, such as a cylindrical lens, not shown in FIG. 2, are also located at this point. In an alternative embodiment, the cylindrical lens may also be of a sliding design and/or serve as an entry window of the disinfection chamber 100. The laser 290 includes a beam shaping element 240 for expanding the laser beam. The laser beam enters the disinfection chamber 100 through a window 210. The laser beam forms a radiation barrier 200 in the disinfection chamber 100, along the longitudinal axis in the central axis of the disinfection chamber 100. In this and the previous embodiment example (FIG. 1), the radiation barrier 200 has a width B corresponding to the width of the disinfection chamber 100. In contrast to the previous embodiment example (FIG. 1), in this embodiment example (FIG. 2c) the entry window 210 is arranged in the brewster angle.

FIG. 2 illustrates the process for disinfecting a gas or liquid. The disinfection chamber 100 has two rows of flow guiding elements 150 arranged parallel to each other, which are arranged opposite each other in such a way that the two rows are displaced from each other by half the width of a flow guiding element 150. The flow guiding elements 150 each have a curved flow guiding surface 160 along which the gas or liquid flow is guided. Radiation barrier 200 is constructed in disinfection chamber 100 to disinfect a gas or liquid. The radiation barrier 200 runs exactly in the middle between the two rows of flow guiding elements 150. Between inlet 170 and outlet 180, the gas or liquid flow is guided through the flow guiding surfaces 160 of the flow guiding elements 150 through the laser barrier 200, advantageously in several points, which increases the time that the laser barrier 200 can act on the substance to be disinfected.

A further embodiment of the disinfection reactor 1 according to the invention is shown in FIG. 4. The disinfection chamber 100 also has two rows of flow guiding elements 150 arranged parallel to each other, which are arranged opposite each other in such a way that the two rows are displaced relative to each other by half the width of a flow guiding element 150. The flow guiding elements 150 each have a curved flow guiding surface 160 along which the gas or liquid flow is guided. In this embodiment, the disinfection chamber 100 has walls 190, 191 on top and bottom sides which are arranged at an angle to the central axis of the disinfection chamber 100. The walls 190, 191 are mirrored such that the laser barrier 200 does not exit the disinfection chamber 100. Alternatively, the walls 190, 191 may act as a beam barrier without being mirrored. The radiation barrier 200 has a width B that follows the course of the walls 190, 191.

FIG. 5 shows another variant of flow guiding elements 150. The disinfection chamber 100 also has two rows of flow guiding elements 150 arranged parallel to each other, which are arranged opposite each other in such a way that the two rows are displaced from each other by half the width of a flow guiding element 150. The flow guiding elements 150 each have a flow guiding surface 160 on the convexly curved side, along which the gas or liquid flow is guided (FIG. 5a). The radiation of the laser 290 is irradiated into the disinfection chamber 100 through a window 210 which is transparent to the radiation. The laser beam enters the disinfection chamber 100 through window 210. The laser radiation forms a radiation barrier 200 in the disinfection chamber 100, which runs along the longitudinal axis in the central axis of the disinfection chamber 100 (FIG. 5b). In the further design, this embodiment example corresponds to the disinfection reactor 1 shown in FIG. 1.

Another embodiment of a disinfection chamber 100 is shown in FIG. 6. The disinfection chamber 100 has a circular cross-section with inner diameter D (FIG. 6a). In the disinfection chamber 100, the flow guiding element 150 is formed by the cylinder wall 110 so that the flow of the fluid or gas passed through the disinfection chamber 150 forms a spiral (vortex) in the longitudinal direction of the disinfection chamber 100 (FIG. 6b). The flow guiding surface 160 is the entire cylinder wall surface 110 of the spiral.

The radiation from the laser 290 is irradiated into the disinfection chamber 100 through a window 210 that is transparent to the radiation. The laser 290 includes a beam shaping element 240 (not shown) that expands and collimates the laser beam in a plane. Optionally, the beam shaping element 240 (e.g. a collimator) may be arranged outside the disinfection chamber 100. Furthermore, the beam shaping element 240 may also be a scanning system. The laser beam enters the disinfection chamber 100 through a window 210. The laser beam forms a radiation barrier 200 in the disinfection chamber 100, which runs along the longitudinal axis in the centre axis of the disinfection chamber 100.

The substance to be disinfected is fed into the disinfection chamber 100 via the inlet 170 in such a way that a continuous gas or liquid flow is formed in the disinfection chamber 100. The substance to be disinfected leaves the disinfection chamber 100 via outlet 180. Between inlet 170 and outlet 180, the gas or liquid flow is swirled by the flow guiding surface 150 (FIG. 6c) and passed several times through the laser barrier 200.

FIG. 3 shows an embodiment of the disinfection chamber 100 according to the invention, in which two radiation barriers 200 are arranged. The disinfection chamber 100 has a circular cross-section with an inner diameter D (FIG. 3c). A flow guiding element 150 is arranged inside the disinfection chamber 100 in such a way that the flow guiding element 150 forms a spiral (vortex) in the longitudinal direction of the disinfection chamber 100 (FIG. 3a). The flow guiding surface 160 is the entire surface of the spiral 150. The radiation of the laser 290 is irradiated into the disinfection chamber 100 through a window 210 that is transparent to the radiation. The laser 290 has a beam shaping element 240 which is used to expand the laser beam. The laser beam enters the disinfection chamber 100 through a window 210. The laser beam forms a primary radiation barrier 200 in the disinfection chamber 100, along the longitudinal axis in the central axis of the disinfection chamber 100. By means of suitable optical components 230, which are arranged on the side of the disinfection chamber 100 opposite the window 210, the radiation barrier 200 is deflected in such a way that it is irradiated into the disinfection chamber 100 at a perpendicular angle (FIG. 3b) to the primary radiation barrier 200 (secondary radiation barrier). This also increases the exposure time of the UV radiation to the substance to be disinfected.

In another embodiment not shown, adjustable flow guiding elements 150 of a Tesla valve can be used to control the flow rate or vortex spread/vortex effective area.

For the radiation, a laser beam with approximately 0.55 mRad beam divergence at 1 mm beam diameter is preferably used. This guarantees an exponentially higher effective range compared to conventional, strongly diverging beam sources, such as gas discharge lamps or LEDs. The power density remains almost the same over the path length. Absorption by air and pathogens is negligible.

To illustrate: The power density of a laser beam at the exit of the beam source 290 is largely unchanged even after several metres distance. If the laser beam is expanded, the beam divergence is reduced according to the expansion factor, so that, for example, even at a distance of 100 meters, the power density is sufficiently high to develop its effect. Consequently, it is irrelevant whether pathogenic substances pass through the laser beam directly at the exit of the beam source 290 or at some distance. If the laser beam is formed into a light plane/light wall 200 by multiple reflection, an effective barrier for pathogenic substances over a large area is ensured. Each passing point in the “beam light wall” 200 experiences the same power density. When using a line optic, such as a Powell lens, the power density of the laser beam is homogeneously distributed.

Preferred is a diffraction-limited laser radiation with a diffraction index M2 close to 1; 0.5 mJ-4 mJ in the higher kHz range with a few nanoseconds pulse duration and a wavelength of 266 nm. Lasers in the femtosecond or picosecond range, which usually produce a pulse peak power in the MW or GW range, as well as continuous lasers can also be used. In our embodiment, a pulse peak power of up to 250 KW is used. A low-cost UV-C laser with a service life of 50000 hours is preferably used.

General advantages of the UV-C laser over conventional UV-C beam sources for inactivating pathogens:

Unsurpassed, high peak pulse powers or high average powers can be generated. The light has a high spatial and temporal coherence. The narrowband nature of the laser light compared to conventional, spontaneously emitting, broadband light from gas discharge lamps, including LEDs, also ensures an increase in the inactivation of viruses.

The possibility of beam shaping of the laser and the resulting area homogeneity and power density is significantly higher compared to ordinary UV-C light.

The laser radiation can be guided to any location and “processed” further.

Laser technology is the only way to disinfect pathogens such as viruses in large public facilities such as hospitals, schools, department stores, airports, hotels, train stations, offices and aeroplanes etc. in a cost-effective and efficient way.—Disinfection in the shortest possible time, with the highest possible water and air flow rate.

### REFERENCE LIST

1 Disinfection reactor
100 Disinfection chamber
110 Side wall
120 Side wall
130 End wall
140 End wall
150 Flow guiding element
160 Flow guiding surface
170 Inlet gas flow/liquid flow
180 Outlet gas flow/liquid flow
190 Wall on top
191 Wall on bottom side
200 Radiation barrier
210 Transparent inlet window
220 Transparent exit window
230 Deflector optics
240 Beam shaping element
290 Radiation element/radiation source
B Width of the radiation barrier
D Diameter of disinfection chamber
Vn Volume range

1. Process for disinfecting liquids or gases, comprising the following process steps:
Creating a radiation barrier (200) in a disinfection chamber (100)
Introduction of a gas and/or a liquid flow into the disinfection chamber (100)
Routing the gas and/or liquid flow through several volume ranges (Vn) of the Disinfection chamber (100)
Discharge of the gas and/or liquid flow from the disinfection chamber (100)
characterised in that
the volume ranges (Vn) in the disinfection chamber (100) are formed by the radiation barrier (200), a chamber wall (110, 120) and a flow guiding surface (160) of a flow guiding element (150), and
the volume ranges are completely separated from each other by the chamber wall (110, 120), the radiation barrier (200) and the flow guiding surface (160).

2. Method for disinfecting liquids or gases according to claim 1
characterised in that
the flow guiding element (150) is arranged as a separate component in the disinfection chamber (100).

3. Method for disinfecting liquids or gases according to claim 1
characterised in that
the flow guiding element (150) is attached to a wall (110, 120) of the disinfection chamber (100).

4. Method for disinfecting liquids or gases according to claim 1
characterised in that
the gas and/or liquid flow is guided through a flow guiding element (150) with several flow guiding surfaces (160) and/or through the flow guiding surfaces (160) of several flow guiding elements (150).

5. Method for disinfecting liquids or gases according to claim 1
characterised in that
the flow guiding surface (160) has a curved surface along which the gas and/or liquid flow is guided.

6. Method for disinfecting liquids or gases according to claim 1
characterised in that
the radiation of the radiation barrier (200) is irradiated into the disinfection chamber (100) through a window (210) transparent to the radiation.

7. Method for disinfecting liquids or gases according to claim 1
characterised in that
the radiation barrier (200) is generated in a width B that is greater than or equal to an inner diameter D of the disinfection chamber (100).

8. Method for disinfecting liquids or gases according to claim 1
characterised in that
the gas and/or liquid flow is guided several times through the radiation barrier (200).

9. Method for disinfecting liquids or gases according to claim 1
characterised in that
the radiation barrier (200) is deflected.

10. Method for disinfecting liquids or gases according to claim 9
characterised in that
multiple radiation barrier sections (Vn) are created in the disinfection chamber (100) as a result of the deflection of the radiation barrier (200).

11. Method for disinfecting liquids or gases according to claim 1
characterised in that
the radiation of the radiation barrier (200) is laser radiation.

12. Method for disinfecting liquids or gases according to claim 1
characterised in that
the entire gas and/or liquid flow is guided through several volume ranges (Vn) of the disinfection chamber (100).

13. Disinfection reactor (1) for disinfecting liquids and/or gases comprising:
a disinfection chamber (100),
the disinfection chamber (100) having an inlet (170) and an outlet (180) and a chamber wall (110, 120),
a flow guiding element (150),
a radiation element (290) adapted to create a radiation barrier (200) in the disinfection chamber (100)
characterised in that
the disinfection chamber (100) has several volume ranges (Vn) completely separated from each other by the radiation barrier (200), the flow guiding element (160) and the chamber wall (110, 120).

14. Disinfection reactor (1) for disinfecting liquids and/or gases according to claim 13
characterised in that
the flow guiding element (150) has a flow guiding surface (160).

15. Disinfection reactor (1) for disinfecting liquids and/or gases according to claim 14
characterised in that
the flow guiding surface (160) has a curved surface.

16. Disinfection reactor (1) for disinfecting liquids and/or gases according to claim 13
characterised in that
the disinfection chamber (100) has a plurality of flow guiding elements (150) and/or flow guiding surfaces (160).

17. Disinfection reactor (1) for disinfecting liquids and/or gases according to claim 13
characterised in that
each volume range (Vn) is separated from an adjacent volume range (Vn) by at least one radiation barrier (200).

18. Disinfection reactor (1) for disinfecting liquids and/or gases according to claim 13
characterised in that
the disinfection chamber (100) has a plurality of radiation barriers (200).

19. Disinfection reactor (1) for disinfecting liquids and/or gases according to claim 13
characterised in that
the disinfection chamber (100) has a transparent window (210, 220) for the radiation generated by the radiation element (290).

20. Disinfection reactor (1) for disinfecting liquids and/or gases according to claim 13
characterised in that
the disinfection chamber (100) comprises elements for deflecting the radiation generated by the radiation element (290).

21. Disinfection reactor (1) for disinfecting liquids and/or gases according to claim 13
characterised in that
the inlet (170) is separated from the outlet (180) by several volume ranges.
